(19) United States
(12) Patent Application Publication
(10) Pub. No.: US 2005/0175644 A1
(43) Pub. Date: Aug. 11, 2005

(54) Preparation for oxidation-sensitive compounds and method for making same

(76) Inventor: Robert Vachy, Versailles (FR)

Correspondence Address: BROWDY AND NEIMARK, P.L.L.C.;624 NINTH STREET, NW, SUITE 300, WASHINGTON, DC, 20001-5303, US

(21) Appl. No.: 10/503,360

(22) PCT Filed: Feb. 4, 2003
(86) PCT No.: PCT/FR03/00386

(30) Foreign Application Priority Data
Feb. 6, 2002 (FR) 02/01456

(51) Int. Cl. A61K 7/00

(52) U.S. Cl. 424/401

(57) ABSTRACT

The invention concerns a method which consists in preventing oxidation of an active oxygen-sensitive compound of a medicinal and/or cosmetic formulation including at least a strong antioxidant and at least a weak antioxidant with high coating capacity, the strong antioxidant reacting with the oxygen before the active compound and the weak antioxidant reacting with the residual oxygen to form, after oxidation, highly coating particles which aggregate by micronization around the active compound to form a protective plaster. The invention is applicable in particular to compounds derived from 2,6-di-tert-butylphenol.

This invention relates to a method to prevent the oxidation of an oxygen-sensitive compound in a medicinal and/or cosmetic preparation. It also relates to a preparation obtained in accordance with the above-mentioned method, as well as to a manufacturing method. It applies in particular, but not exclusively, to 2,6-di-tert-butylphenol derivatives.

Many compounds react with oxygen. These compounds and can lose the properties for which they are used in the course of this oxidation process.

This is the case with 2,6-di-tert-butylphenols which were initially used for their antioxidant property in petroleum products then as food additives because of their effect on animal fats (J. C. Dacre, Biochem J., 1961, vol. 78, no. 4, pp 758-766).

In addition, it is well known that 2,6-di-tert-butylphenol compounds, especially 3,5-di-tert-butyl-4-hydroxytoluene also known as 2,6-di-tert-butyl-4-methylphenol or BHT (“butylated hydroxytoluene”), or its derivatives such as 3,5-di-tert-butyl-4-hydroxybenzoic acid (BG4), octaoxyethyleneglycol 3,5-di-tert-butyl-4-hydroxybenzoate (AVF1) have antiviral properties against lipid-coated viruses (W. Snipes et al., Science, 1975, vol. 288, no. 4183; R. Vachy et al., American Academy of Dermatology, 53^{rd} Annual Meeting, New Orleans, Feb. 4-9 1995; R. Vachy et al., Congress of the Society for Investigative Dermatology, Washington, Apr. 23-27 1997). These properties resulted in their use in medicinal preparations for the treatment of diseases linked to viral infections, in particular infections caused by the herpes virus (FR 2 507 891, EP 0 804 408, WO 91 13626, WO 92 08450).

However, it was found that these compounds lost their antiviral properties as a result of oxidation.

Solutions were therefore put forward to prevent contact between oxygen-sensitive compounds and oxidizing compounds or oxygen-releasing compounds or, quite simply, atmospheric oxygen, for example, by incorporating them into an oil to make up a cream, with the oil acting as an insulating agent.

In addition, packaging in a single-dose gelatine capsule was conceived of in order to protect the cream from air and UV rays until its use.

However, this pharmaceutical form is not the most suitable one in terms of practicality. In fact a cream is poorly absorbed and lives the skin feeling greasy.

Development of a micro-emulsion gave an almost entirely liquid substance that did not have the necessary structure for its intended use.

A method based on multiple phases was found to be far more complicated both in terms of the method employed and the apparatus needed, and with a result that was not always as expected and at a fairly high cost.

Finally, the use of nanoparticles to encapsulate BHT is not satisfactory because this is a complicated, costly process which, in this case, does not allow active products to be encapsulated at the required concentration.

The aim of the invention is to overcome these drawbacks by providing a quick and flexible technique without the risk of oxidation of the active compound.

To this end, it proposes a method which consists in incorporating into a medicinal and/or cosmetic preparation an antioxidant that is more reactive than the active compound so that oxygen is used up before it can react with the active compound, as well as at least one weaker antioxidant which, once oxidized by residual oxygen, acquires a high covering capacity and aggregates around the active compound through micronisation to form a protective layer.

Advantageously, micronisation allows particles to be obtained whose size is between 0.1 and 2 microns, preferably less than 0.2 micron.

The active compound can be a 2,6-di-tert-butylphenol derivative. It can be present in amounts ranging from 1 to 10%, preferably 5%, by weight of the total weight of the preparation.

The preparation can be a dual-phase preparation. In this case, the oily phase can comprise the active compound and the weak antioxidant while the strong antioxidant will be soluble in the aqueous phase.

This combination is of interest insofar as the aqueous phase always contains a certain percentage of oxygen. The strong antioxidant reacts first and, consequently, an residual amount of oxygen crosses the oily phase and contacts with the weak antioxidants. The latter, as they bind to oxygen, become aggregated around the active compounds and form a protective film. This process thus protects the active compound over a long period of time without any changes occurring apart from a minimal change prior to aggregation.

The strong antioxidant can be sodium disulphite. It can be present in an amount ranging from 0.05% to 0.1%, preferably 0.05%, by weight of the total weight of the preparation.

The weak antioxidants can be metal oxides such as oxides of zinc, titanium, aluminium, etc.

The preparation can include two weak antioxidants, for example zinc oxide and titanium dioxide.

Zinc oxide can be present in an amount ranging from 0.1% to 2%, preferably 0.5%, by weight of the total weight of the preparation.

Titanium dioxide can be present in an amount ranging from 5% to 10%, preferably 5%, by weight of the total weight of the preparation.

More generally, the overall amount of weak antioxidant can be between 5.1% and 12%, preferably 5.5%, by weight of the total weight of the preparation.

It should be noted that zinc and titanium oxides have the advantage of possessing anti-UV filtering properties. These properties can be used as protection not only against sunburn but also to prevent herpes in persons predisposed to the infection given that exposure to the sun is known to be a herpes triggering factor.

The preparation can also comprises other compounds with therapeutic properties such as propolis, tepescohuite extracts or even excipients such as antiseptics.

Examples of formulae for the composition will be described hereafter as non-exhaustive examples.

### EXAMPLE 1

### The Composition is as Follows

| Common name | Supplier | Mixture A | % |
|---|---|---|---|
| Cutina CBS | Prod'Hyg | Glyceryl | 4% |
| | | stearate | |
| | | cetearylic | |
| | | alcohol | |
| | | cetyl and | |
| | | cocoglyceride | |
| | | palmitate | |
| Tefose 1500 | Gattefossé | PEG-6*, PEG-32 | 8% |
| | | stearate | |
| Superpolystate | | PEG-6 stearate | 2% |
| Isocetyl | | | 10% |
| stearate | | | |
| Triglycerides | | Caprylic/capric | 6% |
| C8-C10 | | triglycerides | |
| Soya lecithin | Laserson | | 0.1% |
| “Mactan SP65” | | | |
| Common name | Supplier | Mixture A′ | % |
| BHT | | | 5% |
| Zinc oxide | Gattefossé | | 0.5% |
| “Z cote HP1” | | | |
| Titanium | Degussa | | 5% |
| dioxide | | | |
| “T 805” | | | |
| Common name | Supplier | Mixture B | % |
| Demineralised | | | QS** for 100 |
| water | | | |
| Glycerol | | | 2% |
| Common name | Supplier | Mixture C | % |
| Phenonip | SIPCA | phenoxyethanol, | 0.3-0.5% |
| (antiseptic) | | methylparaben, | |
| | | ethylparaben, | |
| | | propylparaben, | |
| | | butylparaben, | |
| | | isobutylparaben*** | |
| Common name | Supplier | Mixture D | % |
| Demineralised | | | 3% |
| water | | | |
| Sodium | | | 0.05% |
| disulphite | | | |

*PEG: polyethylene glycol
**quantity sufficient for 100 g
***paraben: parahydroxybenzoate

Mixture A is heated in a container to a temperature of 70-75° C. Once the temperature stabilizes, components of mixture A′ are added one by one to mixture A with a vigorous stirring (5-10 000 rpm) generated by means of a turbine such that any risk of oxidation of compounds such as BHT linked to the temporary temperature elevation phase are avoided. Moreover, this is when BHT coating by zinc oxide and titanium oxide takes place and it is therefore important that the oil particles are as smallas possible.

Mixture B is heated in a kettle to a temperature of 70-75° C. then added in one go to the reaction mixture (hot oily phase) with slow stirring in order to proceed to the key step, phase inversion.

Micronisation should then be excellent with particles having a size ranging from 0.1 to 2 microns, preferably less than 0.2 micron.

The mixture is then cooled to 60-65° C. under slow stirring then mixture C is added.

The mixture is then brought to a temperature of 40-45° C. as quickly as possible by surrounding the container with a steamer through which water circulates. Mixture D is added then stirring is continued to a temperature of 25-30° C. to produce the desired emulsion, i.e. a very fine, shiny emulsion.

Phase inversion linked to the turbine's rotation rate then a rapid drop in temperature produces BHT coating by zinc and titanium oxides, with a result similar to micro-encapsulation obtained with a multiple emulsion but, in this case, a single step is sufficient instead of a complex operating procedure.

It should be noted that incorporation of compounds other than those mentioned above, for example compounds with additional therapeutic properties, can lead to a variation in the percentages of mixture A in low proportions but sufficient to retain the emulsion's chosen hydrophilic/lipophilic balance.

### EXAMPLE 2

This example makes it possible to produce a cream whose therapeutic properties were described in patent U.S. Pat. No. 6,153,226. It was shown that BHT combined with propolis has enhanced antiviral activity.

The composition is as follows:

| Common name | Supplier | Mixture A | % |
|---|---|---|---|
| Cutina CBS | Prod'Hyg | Glyceryl | 4% |
| | | stearate | |
| | | cetearylic | |
| | | alcohol | |
| | | cetyl and | |
| | | cocoglyceride | |
| | | palmitate | |
| Tefose 1500 | Gattefossé | PEG-6*, PEG-32 | 8% |
| | | stearate | |
| Superpolystate | | PEG-6 stearate | 2% |
| Isocetyl | | | 10% |
| stearate | | | |
| Triglycerides | | Caprylic/capric | 6% |
| C8-C10 | | triglycerides | |
| soya lecithin | Laserson | | 0.1% |
| “Mactan SP65” | | | |
| Common name | Supplier | Mixture A′ | % |
| BHT | | | 5% |
| Zinc oxide | Gattefossé | | 0.5% |
| “Z cote HP1” | | | |
| Titanium | Degussa | | 5% |
| dioxide | | | |
| “T 805” | | | |
| Common name | Supplier | Mixture B | % |
| Demineralised | | | QS** for 100 |
| water | | | |
| Glycerol | | | 2% |
| Common name | Supplier | Mixture C | % |
| Phenonip | SIPCA | phenoxyethanol, | 0.3-0.5% |
| (antiseptic) | | methylparaben, | |
| | | ethylparaben, | |
| | | propylparaben, | |
| | | butylparaben, | |
| | | isobutylparaben | |
| Common name | Supplier | Mixture D | % |
| Demineralised | | | 3% |
| water | | | |
| Sodium | | | 0.05% |
| disulphite | | | |
| Aspartam | | | 0.1% |
| Common name | Supplier | Mixture E | % |
| Liquid | Gattefossé | Aqua and | 0.150% |
| propolis | | propolis wax, | |
| | | phenoxyethanol, | |
| | | methylparaben, | |
| | | ethylparaben, | |
| | | propylparaben, | |
| | | butylparaben*** | |
| Tepescohuite | Laboratoire | Mimosa | 1.0% |
| glycolic | Mu | Tenuiflora | |
| extract | | | |
| Passion fruit | Agipal | Passion flower | 0.2% |
| flavouring | | fragrance | |
| | | (Passiflora | |
| | | carnata) | |

*PEG: polyethylene glycol
**quantity sufficient for 100 g
***paraben: parahydroxybenzoate

In the same way as in example 1, mixture A is heated in a container to a temperature of 70-75° C. Once the temperature stabilizes, components of mixture A′ are added one by one to mixture A with a vigorous stirring (5-10 000 rpm) generated by means of a turbine such that any risk of oxidation of compounds such as BHT linked to the temporary temperature elevation phase are avoided.

Mixture B is heated in a kettle to a temperature of 70-75° C. then added in one go to the reaction mixture (hot oily phase) with slow stirring in order to proceed to phase inversion.

The mixture is then cooled to 60-65° C. under slow stirring then mixture C is added.

The mixture is then brought to a temperature of 40-45° C. as quickly as possible by surrounding the container with a steamer through which water circulates and mixture D is added.

The mixture continues to be cooled and, at 35° C., the compounds of mixture E are added then stirring is continued to a temperature of 25-30° C. to give the desired emulsion, i.e. a very fine, shiny emulsion.

Skin and eye tolerance studies were conducted on the cream obtained in this manner.

The first acute skin tolerance study was conducted on a group of ten adult volunteers by a single application to the skin of the inner side of the forearm under an airtight dressing for 48 hours. This test was performed according to the methodology applied to airtight epicutaneous tests.

The group included seven female volunteers and three male volunteers aged between 19 and 36 years with no history of intolerance or allergy to a cosmetic product, no history of skin disease and not taking any medication likely to disrupt skin metabolism.

The product was applied pure in a single application to a 50 mm² area of skin on the inner side of the forearm of each volunteer at a dose of about 0.02 ml on a circular piece of filter paper held in place by the airtight dressing.

The product was kept in contact with skin for 48 consecutive hours.

This application was carried out in parallel to and under the same conditions with a test airtight dressing only (no product) as a negative control.

Macroscopic skin examinations were performed immediately, 30 minutes after removal of the airtight dressing.

Assessment of skin reactions (erythema, oedema, etc.) was based on the nomenclature suggested by the International Contact Dermatitis Research Group (I.C.D.R.G.):
NT: not tested
?+: doubtful reaction: slight erythema only
+: weak positive reaction (non-vesicular): erythema, infiltration, a few papules at times,
++: Strong positive reaction: erythema, papules, vesicles present
+++: Violent positive reaction, with blisters
−: Negative reaction
IR: Irritation reaction:
E 0.5: very mild erythema
E1: mild erythema
E2: clear erythema
E3: substantial erythema

The test was stopped if no local skin reaction was observed at the 30-minutes reading after removal of the dressing. Nevertheless, each volunteer was asked to check for the absence of any reaction the next day. In the event of a visible reaction, the volunteer was asked to come back to the centre.

In the case of clear or doubtful reactions, a reading was performed 48 hours and, where necessary, 72 hours after dressing treatment.

Interpretation of the results was carried out as outlined below (“Les Essais Cliniques en Dermatologie”, Therapie, 1991, Vol 46, pages 183-187):

The average irritation index at each reading time was calculated according to the ratio:
A.I.I.=Σerythema scores/number of volunteers

The scale interpretation of skin irritation is as follows:
If A.I.I.<0.20: non-irritant
If 0.20≦A.I.I.<0.50: mildly irritant
If 0.50≦A.I.I.<1: moderately irritant
If A.I.I.≧1: irritant

The results are grouped together in table I below

TABLE I

| | | Product tested | | Negative control | |
|---|---|---|---|---|---|
| VOLUNTEERS | | Reading | Reading | Reading | Reading |
| | Age | 30 min | 24 h | 30 min | 24 h |
| | and | after | after | after | after |
| | gender | dressing | dressing | patch | patch |
| Identification | (1) | removal | removal | removal | removal |
| MA.LA | 19 F | — | — | — | — |
| NE.SA | 25 F | — | — | — | — |
| DE.JE | 22 M | — | — | — | — |
| PO.JO | 20 M | — | — | — | — |
| PE.GU | 24 F | *0.5 | — | — | — |
| DO.FA | 33 F | — | — | — | — |
| BA.RA | 19 F | — | — | — | — |
| AT.PH | 30 M | — | — | — | — |
| SU.VE | 36 M | — | — | — | — |
| LA.DE | 20 F | — | — | — | — |

Average A.I.I.

| | 0.05 | 0 | 0 | 0 |
|---|---|---|---|---|
| Results | Non | Non | Non | Non |
| | irritant | irritant | irritant | irritant |

*Volunteer 5: skin dryness accompanied by slight peeling
(1): M = male F = female

Under the retained experimental conditions, one volunteer showed skin dryness accompanied by mild peeling and very mild erythema at the site of application 30 minutes after removal of the dressing.

24 hours and 48 hours after removal of the dressing, this volunteer's skin was normal and no side effect was observed.

In conclusion, the pure product applied locally under an airtight dressing for 48 hours to the skin of ten adult volunteers was found to be non irritant.

Acute ocular tolerance was also evaluated on reconstituted corneas (SKINETHIC model) and on rabbit eyes.

To start with, the product was evaluated in terms of its capacity to trigger cytopathic effects on corneas reconstituted by means of in vitro transformed keratinocytes cultures.

Cell cultures are generally regarded in the scientific literature as a highly sensitive and reliable method for the study of pharmaceutical, cosmetic or medical products.

When they are cultured at the air-liquid interface in a specific medium, transformed human keratinocytes (cell line TR 146) form epithelial tissue without a corneal layer, similar to the human eye.

A sample of the product to be studied (30 μl) was deposited on each of the six equivalent reconstituted corneal cultures and spread out using a thin brush.

Two cultures were then incubated at 37° C., 5% CO₂ for 10 minutes, 1 hour, 3 hours and 24 hours.

Negative controls substances (buffered phosphate saline solution A) and positive controls substances (0.4% SDS for “sodium dodecyl sulfate” in solution A) were prepared under sterile conditions and deposited simultaneously on the other two cultures. These cultures were incubated for one hand 24 hours.

A culture containing no treatment and acting as a negative control was incubated in parallel.

The viability or death of cultures basal layer keratinocytes was tested by means of a MTT cell viability test.

Cell viability was measured qualitatively after staining.

The MTT system measures the mitochondrial dehydrogenase activity of living cells. The key compound is 3-[4,5-dimethylthiazole-2-yl]-2,5-diphenyl tetrazolium bromide (MTT).

MTT buffered saline solutions, in the absence of phenol red, are yellow. The mitochondrial dehydrogenase of living cells breaks the tetrazolium cycle and triggers the formation of purple MTT formazan crystals, insoluble in aqueous solutions.

The crystals formed by viable cells are trapped by polycarbonate filters acting as supports for epithelial cultures.

The cultures all turn a deep blue/purple colour when they are viable but are white/yellow in colour if cell death occurs.

The results are compared with the negative and positive controls substances.

In practice, 0.15 ml of culture medium containing 10% vol/vol of MTT solution is added in the form of culture filters/supports.

After incubation for 30 minutes at room temperature, each culture's colour is observed and recorded:
negative control cultures should be a deep blue/purple colour, evidence of the viability of cells in the basal layer after 24 hours in contact,
positive control cultures should be white, evidence of cell death from the first hour of contact.

Interpretation of the results is as follows:
non-irritant (NI): blue at 10 minutes, 1 hour, 3 hours and 24 hours,
very mildly irritant (VMI): blue at 10 minutes, 1 hour, 3 hours and white at 24 hours,
mildly irritant (MI): blue at 10 minutes, blue/white at 1 hour md 3 hours and white at 2 hours,
irritant (I): 24 hours,
very irritant (VI): white at 10 minutes, 1 hour, 3 hours and 24 hours.

The results are grouped together in table II below:

TABLE II

| | Colour of the two cultures | | | | |
|---|---|---|---|---|---|
| Product | 10 min. | 1 hour | 3 hours | 24 hours | Toxicity |
| Cream | blue | blue | blue | white | VMI |
| Negative | — | — | — | blue | NI |
| control | | | | | |
| Positive | — | white | — | — | VI |
| control | | | | | |

Cell viability of cells making up the corneal layer is intact after 10 minutes, 1 hour, 3 hours of contact with the pure product studied. After 24 hours, there is near-total cell death.

In conclusion, in pure form, the studied cream is very mildly irritant to cells used to make up the in vitro “reconstituted cornea” model.

This study was completed by an additional control test based on the method described in the “Journal Officiel de la République Française” dated Jun. 9, 1992, on rabbit eyes.

A single rabbit was used

One hour after instilling the pure product studied, no ill effects were observed: the rabbit eye was normal.

In view of these results, the product can be retained as mildly irritant for rabbit eyes.

Consequently, given the set of results obtained under different experimental conditions, the cream tested and put in contact with the eye does not present a risk to the eyes. It can therefore be retained as mildly irritant.

Moreover, the conducted tests and studies show that this product contains no starting material whose use is banned, that it complies with standards, especially with regard to contaminants, and that it complies with European Pharmacopoeia guidelines in terms of the efficacy of antimicrobial agents.

The tests conducted under acute conditions show that under normal conditions of use, the product should not cause any particular intolerance reaction.

Finally, the stability of BHT in a preparation according to the invention was tested:
a container with 40 kg of preparation, covered with a simple lid without any special packaging such as the addition of an inert gas (e.g. nitrogen), was placed in storage for three yeas.

Analysis by liquid chromatography carried out after three years showed that BHT concentrations, taking into account the error coefficient for the employed technique, remained the same.

Consequently, the stability of BHT in a preparation according to the invention is excellent.

1. Method to prevent the oxidation of an oxygen-sensitive active compound in a medicinal and/or cosmetic preparation,
consisting of incorporating at least one strong antioxidant and at least one weak antioxidant with a high coating capacity, such that the strong antioxidant reacts with oxygen before the active compound and the weak antioxidant reacts with residual oxygen to form, after oxidation, particles with a high coating capacity which aggregate through micronisation around the active compound to form a protective layer.

2. Medicinal and/or cosmetic preparation containing an oxygen-sensitive active compound whose oxidation is to be prevented,
comprising, in addition to said active compound, at least one strong antioxidant and at least one weak antioxidant with a high coating capacity by micronisation.

3. Preparation according to claim 2,
wherein the active compound is a derivative of 2,6-di-tert-butylphenol.

4. Preparation according to claim 2,
wherein said active compound is present in amounts ranging from 1 to 10%, preferably 5%, by weight of the total weight of the preparation.

5. Preparation according to claim 2,
wherein the weak antioxidant is a metal oxide.

6. Preparation according to claim 5,
wherein said weak antioxidant is present in overall an amount ranging from 5.1 to 12%, preferably 5.5%, by weight of the total weight of the preparation.

7. Preparation according to claim 2,
comprising two weak antioxidants.

8. Preparation according to claim 7,
wherein the two antioxidants are zinc oxide present in an amount ranging from 0.1% to 2%, preferably 0.5%, by weight and titanium dioxide present in an amount ranging from 5% to 10%, preferably 5%, by weight of the total weight of the preparation.

9. Preparation according to claim 2,
wherein the strong antioxidant is sodium disulphite.

10. Preparation according to claim 9,
wherein the strong antioxidant is present in an amount ranging from 0.05% to 0.1%, preferably 0.05%, by weight of the total weight of the preparation.

11. Preparation according to claim 2,
wherein said preparation is a dual-phase preparation, an oily phase and an aqueous phase.

12. Preparation according to claim 2, wherein the preparation has particles having a size between 0.1 and 2 microns, preferably less then 0.2 micron.

13. Preparation claim 2, having an oily phase which comprises the active compound and the weak antioxidant.

14. Preparation according to claim 2,
having an aqueous phase which comprises the strong antioxidant.

15. Preparation according claim 2,
comprising five mixtures:
mixture A comprising glyceryl stearate, cetearylic alcohol, cetyl palmitate and cocoglyceride, PEG-6 stearate, PEG-32 stearate, triglycerides,
mixture A′ comprising the above-mentioned active compound and above-mentioned weak antioxidant,
mixture B comprising of demineralised water and glycerol,
mixture C comprising antiseptics,
mixture D comprising demineralised water and above-mentioned strong antioxidant.

16. Preparation according to claim 2,
comprising six mixtures:
mixture A comprising glyceryl stearate, cetearylic alcohol, cetyl palmitate and cocoglyceride, PEG-6 stearate, PEG-32 stearate, triglycerides,
mixture A′ comprising the above-mentioned active compound and above-mentioned weak antioxidant,
mixture B comprising demineralised water and glycerol,
mixture C comprising antiseptics,
mixture D comprising demineralised water and above-mentioned strong antioxidant.
mixture E comprising propolis and tepescohuite glycolic extract.

17. Method for the manufacture of a preparation for oxygen-sensitive compounds comprised at least one active compound, at least one strong antioxidant and at least one weak antioxidant, such that the strong antioxidant reacts with oxygen before the active compound and the weak antioxidant reacts with residual oxygen to form particles with a high coating capacity which aggregate through micronisation around the active compound to form a protective layer,
wherein:
a) mixture A is heated to 70-75° C. then the components of mixture A′ are added one by one,
b) mixture B is heated to 70-75° C. then added to the reaction mixture,
c) the entire mixture is cooled to 60-65° C. then mixture C is added,
d) the mixture is adjusted to a temperature of 40-45° C. and mixture D is added,
e) where appropriate, mixture E is added at 35° C.,
f) stirring is continued until the temperature drops to around 25-30° C.
